(19) United States
(12) Patent Application Publication
(10) Pub. No.: US 2003/0143592 A1
(43) Pub. Date: Jul. 31, 2003

(54) DNA chip

(75) Inventors: Koichi Kawamura, Shizuoka-ken (JP); Yoshihiko Makino, Saitama-ken (JP); Miki Takahashi, Shizuoka-ken (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/278,083

(22) Filed: Oct. 23, 2002

(30) Foreign Application Priority Data
Oct. 25, 2001 (JP) 2001-327569
Oct. 26, 2001 (JP) 2001-329170

(51) Int. Cl. C12Q 1/68; C12M 1/34

(52) U.S. Cl. 435/6; 435/287.2

(57) ABSTRACT

The present invention provides a DNA chip including: a solid support having a surface; a DNA fragment immobilized on the surface; and a graft polymer bonded to the surface, wherein the DNA fragment is immobilized on the surface via the graft polymer. The invention also provides a DNA chip including: a solid support made of a resinous material and having a surface; a DNA fragment immobilized on the surface; and a graft polymer bonded to the surface, wherein the DNA fragment is immobilized on the surface via the graft polymer.

### BACKGROUND OF THE INVENTION

1. Field of the Invention

The present invention relates to a DNA chip, and more specifically to a DNA chip that is used in genetic analysis and the like, and has high DNA-adsorbing efficiency enabling immobilization of a large amount of DNA fragments per unit area of the chip. Further, the invention relates to a DNA chip that is light-weight and is excellent in processability.

2. Description of the Related Art

A DNA chip is attracting an attention as a means for efficiently analyzing the function of genes in an organism. Usually, a DNA chip has a form in which many DNA fragments are arrayed and immobilized on a solid support such as a slide glass. The DNA chip is used, e.g., for detecting a DNA fragment (target) contained in a sample by capturing the target fragment, which is complementary to another DNA fragment (probe) immobilized on the DNA chip, by effecting hybridization between the two fragments to thereby bind the target fragment to the chip.

Since this immobilizing technique can also be applied to bio-molecules other than DNA, the technique is expected to provide a new means for research in drug-design, development of diagnostic and preventive methods of diseases, and measures to cope with energy and environmental problems.

Heretofore, various methods of immobilizing DNA fragments on the surface of the solid support have been studied. Most of the solid supports conventionally used were made of glass. Glass has the advantage of being superior for detection of fluorescent signals after hybridization because it is non-fluorescent and has a high flatness. Thus, glass is an excellent substrate for research use. A representative method is a bonding method, in which aminopropylsilane is allowed to react with a surface of the glass substrate, an amino group therein bonds with the surface of the substrate, and the DNA fragment bonds on the substrate via this amino group.

For example, Japanese Patent Application Laid-Open (JP-A) No. 2001-178459 specifically discloses a technique in which maleic anhydride is allowed to react with the amino groups attached to the glass surface to prepare a maleimide-coated glass and the resultant coated glass is then reacted with DNA fragments, into which a furfurylamino group has been introduced, to bond the DNA to the glass substrate via covalent bonding. JP-A No. 2001-178466 discloses another technique in which one end of a linking agent such as chloroisocyanate or succinimidyl(4-vinylsulfonyl) benzoate, which is a joining agent carrying two reactive groups, is allowed to react with amino groups attached to the glass surface, then the other end is covalently bonded to amino groups which have been introduced into DNA fragments.

In addition to the covalent bonding described above, DNA fragments and the surface of the solid support may be bonded via ionic bonding, hydrogen bonding, hydrophobic bonding, etc. For example, JP-A No. 2001-178458 discloses a technique in which DNA fragments are bonded to the surface of the support via host-guest interaction.

Although the aforementioned DNA chips can immobilize a sufficient amount of DNA fragments necessary for detecting a target fragment, there is still a demand for a technique enabling immobilization of an increased amount of DNA fragments in a given area, from the viewpoint of increasing detecting sensitivity to meet the expected levels required in genetic analysis and the like. Namely, the current situation is that in these known immobilizing methods, the desired reactions do not all progress sufficiently, thereby leaving a part of the solid support unreacted and failing to immobilize some of DNA fragments.

When a detection method is implemented, in which method a target fragment, which is contained in the sample and complementary to another DNA fragment (probe) immobilized on the DNA chip is detected via hybridization between the two fragments, it is desired to increase effective hybridization of the labeled DNA, relative to a predetermined amount of the probe fragments immobilized on the surface of the support. However, in the DNA chips produced by conventional methods, the ratio of immobilizing DNA fragments on the support and the hybridizing efficiency are not always high. Therefore, a DNA chip improved in detecting sensitivity is still demanded.

In recent years, use of DNA chips is widespread, and large amounts of DNA chips are utilized in genetic diagnosis, etc. When used, e.g., in the diagnostic field, the DNA chip has a problem in that the slide glass is heavy and liable to break. Further, due to its nonflammability, it is difficult to dispose of the slide glass after its use. Accordingly, a DNA chip having increased workability and easier disposal in practical use is needed. Further, from the viewpoint of high producibility, there is a need for a novel substrate for the DNA chip which allows large-area immobilization of DNA fragments on the support at one time and has superior processability.

Because of this background, various tests have been performed to utilize a plastic film in place of glass. However, when the plastic film is used as the solid support, an effective method to bind reactive functional groups which serve to attach DNA fragments to the plastic film surface has not been found. There has been proposed, for example, a method in which a coating solution containing a polymer carrying reactive functional groups is applied to a plastic film surface and then crosslinking is performed to form a crosslinked layer, after which DNA fragments are linked via the reactive functional groups that are present in the crosslinked layer and serve as the starting point of binding. However, this method has a problem in that although the crosslinked layer having a crosslinked structure acquires increased strength, the layer has poor adherence to the plastic film and hence is easily peeled from the support surface. Further, this method has another problem in that when the polymer having reactive functional groups undergoes crosslinking, a part of the reactive functional groups is buried in the crosslinked polymer layer, whereby the number of reactive sites of DNA fragments is reduced, resulting in a lowered DNA immobilizing efficiency.

### SUMMARY OF THE INVENTION

In view of the problems of the prior art described above, an object of the present invention is to provide a DNA chip that has high DNA-adsorbing efficiency enabling immobilization of a large amount of DNA fragments per unit area of the chip. Another object of the invention is to provide a DNA chip that is useful in analyzing the function of genes in an organism and capable of binding a target DNA fragment via highly efficient hybridization, the target DNA fragment being complementary to another DNA fragment (probe) immobilized on the support of the DNA chip. Further, another object of the invention, particularly in a case where a solid support made of a resinous material is used, is to provide a DNA chip, which is able to immobilize a large amount of DNA per unit area and has active spots capable of adsorbing DNA at a high density.

Through extensive research, the present inventors found that DNA can be immobilized densely and efficiently on the surface of the solid support via a graft polymer chain and accomplished the present invention. The inventors also found that the above-described problems can be solved by incorporating functional groups that are highly reactive with DNA into the surface of the solid support made of a resin, via the graft polymer chain, and achieved the invention.

A first aspect of the invention is a DNA chip comprising: a solid support having a surface; a DNA fragment immobilized on said surface; and a graft polymer bonded to said surface, wherein the DNA fragment is immobilized on said surface via said graft polymer.

A second aspect of the invention is a DNA chip comprising: a solid support made of a resinous material and having a surface; a DNA fragment immobilized on said surface; and a graft polymer bonded to said surface, wherein the DNA fragment is immobilized on said surface via said graft polymer.

Although the action of the DNA chip of the invention is not clear, it is thought to be the following. In the DNA chip according to the first aspect of the invention, DNA fragments are bonded to the surface of the solid support via a graft polymer chain. Since the one end of the polymer chain is firmly bonded to the surface of the solid support, and since the active sites, which contribute to the bonding of DNA fragments at the middle of the polymer chain, are comprised at a high density relative to unit area of the solid support, it is thought that the DNA chip of the invention will be able to immobilize the DNA fragments densely. Moreover, since the portions of the graft chains having reactive functional groups are not crosslinked with each other, the graft polymer chain maintains a state of high motility and moves freely in a solvent, it is thought that reactivity between the reactive functional groups in the graft polymer chain and the DNA fragments will be enhanced to achieve highly efficient DNA immobilization.

Even when these DNA fragments, which have a high degree of freedom, are immobilized on the DNA chip, it is thought that the immobilized DNA fragments (probe) will be able to perform hybridization with high efficiency, because the probe fragments express a high reactivity towards the complementary (target) fragments contained in a sample.

The DNA chip according to the second aspect of the invention has a structure in which a polymer having a reactive functional group is grafted to a plastic film. Thus, a reactive substrate can be obtained, in which reactivity of immobilizing the DNA fragments is not reduced and which has excellent adhesion. In particular, good binding can be obtained by graft-polymerizing a monomer having reactive functional groups to the surface of the plastic film such that the graft polymer is directly linked to the surface of the solid support via covalent bonding. In a DNA chip having this structure, the graft chain portions in the polymer, which have reactive functional groups capable of interacting with the DNA fragments, are not crosslinked to each other and exhibit high motility and move freely in the solvent. Thus, it is thought that since reactivity between the reactive functional groups in the graft polymer chain and the DNA fragments is enhanced, highly efficient DNA immobilization can be achieved.

### DESCRIPTION OF THE PREFERRED EMBODIMENTS

The present invention will now be explained in more detail below.

Solid Support

A solid support, which forms a DNA chip, is not particularly limited and any solid support may be suitably selected and used depending on the intended purpose.

In the first aspect of the invention, from the standpoint of selectively immobilizing DNA fragments on a desired site, a support exhibiting hydrophobicity or low hydrophilicity at the surface thereof is used. A support, which has a smooth surface or a support, which has a low smoothness due to unevenness, may be preferably used.

Examples of the material for the solid support include inorganic materials including inorganic amorphous materials such as glass, cement, ceramics and new ceramics; silicon used for an Si substrate; inorganic materials such as activated carbon; and conductive materials such as gold. The shape of the support is generally a plate, but is not limited thereto. The support may be a porous substance such as porous glass, porous ceramics, porous silicon, porous activated carbon or a membrane filter. The support may have the shape of a fabric, a knit, a non-woven fabric or the like produced by using long fibers or short fibers made of any material listed above. The area of the solid support surface can be increased by using a material having minute roughness on the surface thereof or a porous substance. When a porous substance is used, the size of fine pores is preferably from 2 to 1,000 nm, and more preferably from 2 to 500 nm.

The material for the solid support of the DNA chip according to the first aspect of the invention is preferably glass or silicon, from the standpoints of easily conducting surface treatment and readily performing analysis using electro-chemical method. Generally, the solid support has a thickness of 100 μm to 2000 μm, depending on the use purposes of the DNA chip.

In the second aspect of the invention, a resinous material such as a plastic film is used as the solid support. The plastic film for use as the solid support may be a conventionally known film. In view of physical properties, the material having high strength and excellent flatness is desirable. When DNA is analyzed using a reagent containing fluorescents, it is preferable to use a non-fluorescent film.

In view of high strength, a film called an engineering plastic may generally be used. Further, any film described in Engineering Plastics, revised 3rd edition, 1985 (published by The Chemical Daily Co., Ltd.) may be used.

Examples of the film suitably used in the invention include cellulose acetate butyrate, cellulose triacetate, cellulose tributyrate, polyacetal, polyamide (including aliphatic polyamides and aromatic polyamides such as aramid), polyamideimide, polyarylate, polyimide, polyetherimide (PEI), polyetheretherketone (PEEK), polyethersulfone (PES), polyethylene terephthalate (PET), polyethylene naphthalate (PEN), polycarbonate, polysulfone (PSF), polystyrene, polycellulose triacetate, polyphenylene sulfide (PPS), polyphenylene oxide (PPO), polybenzoxazole and amorphous polyacrylate (PAR).

As the non-fluorescent plastic film, polymers having no aromatic group in the polymer skeleton are preferably used. Specific examples thereof include polymethyl methacrylate, cellulose acetate butyrate, cellulose triacetate, cellulose tributyrate, polyacetal and polyamide.

Additional resinous materials, usable as the solid support, include synthetic resins such as an epoxy resin, an acrylic resin, an urethane resin, a phenolic resin, a styrene-type resin, a vinyl-type resin, a polyester resin, a polyamide-type resin, a melamine-type resin and a formalin resin.

The DNA chip according to the second aspect of the invention comprises a solid support made of a resinous material and having a graft polymer introduced into the surface thereof, and a reactive functional group present in a side chain of the graft polymer is allowed to interact with a DNA fragment to thereby immobilize the fragment on the support. Since the solid support is made of the resinous material, the DNA chip is light-weight and has high impact resistance, excellent processability, good workability, and easiness in disposal after its use.

Graft Polymer

The graft polymer used in the invention for bonding the DNA fragments to the solid support is not particularly limited insofar as the graft polymer has in a side chain thereof reactive functional groups capable of bonding with DNA fragments.

Examples of the reactive functional group include a carboxyl group, an amino group, a hydroxyl group, an epoxy group, an acid anhydride group, a sulfonic acid group, an isocyanate group, a trialkoxysilyl group, a thioisocyanate group, a phosphoric acid group and a phosphonic acid group. The functional groups that are derived from the above-mentioned functional groups may also be used. Examples of the functional groups derived from the carboxyl group include an acid anhydride, an active ester of N-hydroxysuccinimide, benzyl alcohol or the like, and a carboxyl halide. Examples of the functional groups derived from the sulfonic acid group include an active ester of benzyl alcohol, and sulfonyl halide.

Method for Immobilizing DNA Fragment Via Graft Polymer

It is possible to immobilize DNA fragments utilizing reactive functional groups present in the side chain of the graft polymer by employing a conventionally known method. For example, JP-A No.2001-178460 discloses a method for immobilizing DNA fragments using a solid support to which a polymer, comprising as the constituent unit a monomer carrying a carboxyl group, is bonded. When this method is employed, the solid support bearing (binding) an acrylic acid-grafted polymer is treated with a dehydrating agent to alter the terminal end to become a cyclic acid anhydride to be utilized as the reactive functional group. Then an aqueous liquid containing DNA fragments having a terminal amino group is applied spot-wise, to cause interaction between the DNA fragments and the produced reactive functional group such that the DNA fragments can be immobilized on the support.

When the solid support to which the acrylic acid-grafted polymer is bonded is used, carboxylic acid is converted into an active ester which acts as a reactive functional group, using a water-soluble carbodiimide. Thus, DNA fragments can be immobilized by spot-wise application of the aqueous liquid containing DNA fragments having an amino group.

In a preferred embodiment of the invention, DNA having a known base sequence and carrying a terminal amino group is used and as the reactive functional group, a group capable of covalently binding to the amino group is selected and used in the DNA chip. In order to obtain functional groups having higher reactivity, it is preferable to convert carboxylic acid into an acid anhydride or an active ester.

Conventionally known reagents may be used as the reagent for converting carboxylic acid into the acid anhydride or the active ester. Specific examples of the reagent include acid anhydrides (e.g., acetic anhydride and propionic anhydride), acid halides (e.g., methanesulfonyl chloride and isobutyl chlorocarbonate), carbodiimides (e.g., dicyclohexylcarbodiimide and 1-ethyl-3-(3-dimethylaminopropyl)carbodiimide hydrochloride), 2-halopyridinium compounds (e.g., 2-fluoro-1-methylpyridinium iodide), 1,1,3,3-tetraalkylamidinium compound having a leaving group at the 2-position (e.g., o-(benzotriazole-1-yl)-N,N,N′,N′-tetramethyluronium hexafluorophosphate and 2-chloro-1,3-dimethylimidazolinium chloride), and active esters and active amides (e.g., 1-succinimidyl acetate and carbonyldiimidazole).

The method for introducing such graft polymers into the surface of the solid support will be explained below.

The solid support used in the invention is characterized in that a graft polymer chain is bonded to the surface of the solid support. The graft polymer chain may be bonded directly to the surface of the solid support. Alternatively, an interlayer to which the graft polymer is easily bonded is provided on the surface of the solid support so that the polymer having a reactive functional group at the terminal end can be grafted to the interlayer. Further, in order to bind a graft polymer to the solid support, a polymer with the graft polymer chain grafted onto the backbone polymer is coated on the surface of the solid support, or a polymer with the graft polymer chain grafted onto the backbone polymer and additionally having a crosslinkable functional group introduced thereto is coated on the surface of the solid support, and crosslinking is further performed after the coating. Alternatively, a composition containing a hydrophilic polymer carrying a terminal crosslinkable group and a crosslinking agent is coated on the surface of the solid support, and crosslinking is further performed after the coating.

The graft polymer for use in the invention is characterized in that one end of the polymer is bonded to the surface of the solid support or to the surface layer of the solid support and in that the graft portion where the reactive functional group capable of interacting with DNA fragments is present has substantially un-crosslinked. Because of this structure, mobility of the polymer at the portion having the reactive functional group capable of bonding with DNA is not restricted; or the polymer at this portion can maintain high motility without being buried in a tightly crosslinked structure. For this reason, it is thought that the graft polymer having a large number of sites, that are reactive with DNA, achieves good bonding with DNA, as compared with a hydrophilic polymer having an ordinary crosslinked structure and carrying a reactive functional group within the molecule.

The molecular weight (Mw) of the graft polymer chain is 500 to 5,000,000, preferably of 1000 to 1,000,000, and more preferably of 2000 to 500,000.

As used herein, (a) the structure in which the graft polymer chain is bonded directly to the surface of the solid support or to the interlayer provided on the surface of the solid support is referred to as “surface graft”, and (b) the structure in which the graft polymer chain is introduced into the crosslinked polymer layer is referred to as “crosslinked layer to which graft chains are introduced”. Further, as used herein, the solid support or the solid support provided with an interlayer may occasionally be referred to as “substrate”.

(a) Method for Producing Surface Graft

A surface having reactive functional groups of the graft polymer may be formed on the substrate mainly by two methods: one is a method in which the graft polymer is linked to the substrate via chemical bonding; and the other is a method in which a compound having a polymerizable double bond is allowed to polymerize at the starting point on the substrate to form a graft polymer.

First, a method of attaching a graft polymer to the substrate via chemical bonding is described.

In this method, a polymer having at the terminal or the side chain thereof a functional group is used to cause a chemical reaction between this functional group and the functional group on the surface of the substrate to make the polymer grafted on the substrate. The functional group which is reactive to the substrate is not particularly limited so long as it is reactive to the functional group on the surface of the substrate. Examples of the functional group include a silane coupling group such as alkoxysilane, an isocyanate group, an amino group, a hydroxyl group, a carboxyl group, a sulfonic acid group, a phosphoric acid group, an epoxy group, an allyl group, a methacryloyl group and an acryloyl group. Particularly useful compounds as the polymer having the functional group at the terminal or the side chain thereof include a polymer having a trialkoxysilyl group at the polymer terminal, a polymer having an amino group at the polymer terminal, a polymer having a carboxyl group at the polymer terminal, a polymer having an epoxy group at the polymer terminal, and a polymer having an isocyanate group at the polymer terminal.

The polymer for use in the invention is not particularly limited so long as it has a reactive functional group. Specific examples thereof include polyacrylic acid, polymethacrylic acid, polystyrene sulfonic acid, poly-2-acrylamide-2-methylpropane sulfonic acid and the salts thereof, polyacrylamide and polyvinyl acetamide. Additionally, the polymers synthesized from the monomer or the copolymers containing the monomer, used for surface graft polymerization described below, may be advantageously used.

A graft polymer is generally produced by a method called surface graft polymerization in which a compound having a polymerizable double bond is allowed to polymerize from the starting point on the substrate. In this surface graft polymerization, active species are provided on the surface of the substrate by conducting plasma irradiation, light irradiation or heating to make the compound, having the polymerizable double bond and arranged to contact with the substrate, to bind to the substrate through polymerization.

Any known method described in literatures may be used for the surface graft polymerization in the invention. For example, as the surface graft polymerization, photo-graft polymerization and plasma-graft polymerization are described in New Polymer Experimental Studies 10 (edited by The Society of Polymers, Japan), 1994, published by Kyoritsu Shuppan Co., Ltd., p. 135. Further, radiation-graft polymerization using a y-ray, an electron beam or the like is described in Adsorption Technology Handbook, supervised by Takeuchi, NTS Co., Ltd., issued in February 1999, p. 205 and p. 695. The methods disclosed in JP-A Nos.63-92658, 10-296895, and 11-119413 may be used as specific examples of the photo-graft polymerization. As plasma-graft polymerization and radiation-graft polymerization, the methods described in the above literatures and the method described, for example, in Ikada et al., Macromolecules, Vol. 19, page 1804(1986) may be employed.

More specifically, the surface of a polymer such as PET or the surface of a solid support made of a resinous material is treated with a plasma or an electron beam so as to generate radicals on the surface and the resultant activated surface is allowed to react with a monomer having a reactive functional group to produce a surface layer to which a graft polymer is bonded.

In addition to the disclosure contained in the above literatures, photo-graft polymerization can be carried out by a process which comprises coating on the surface of a film-base material a photo-polymerizable composition, contacting with an aqueous radical-polymerizable compound and irradiating the layer with light as disclosed in JP-A No.53-17407 (Kansai Paint Co., Ltd.) and JP-A No.2000-212313 (Dainippon Ink and Chemicals, Incorporated).

Compound Having a Polymerizable Double Bond Useful for Surface Graft Polymerization

The compound having a polymerizable double bond useful for forming a graft polymer chain is required to have the polymerizable double bond and to carry a reactive functional group within the molecule. Any compound including a polymer, an oligomer or a monomer may be used for this purpose so long as it contains a double bond in the molecule. A particularly useful compound is the monomer carrying the reactive functional group.

Examples of the monomer carrying the reactive functional group usable in the invention include a monomer having positive charges, such as ammonium and phosphonium, or a monomer containing acidic groups having negative charges or capable of dissociating into negative charges, such as sulfonic acid groups, carboxyl groups, phoshoric acid groups and phosphonic acid groups. Additional monomers having a non-ionic group such as a hydroxyl group, an amide group, a sulfonamide group, an alkoxy group, and a cyano group may be used.

Specific examples of the monomer carrying the reactive functional group usable in the invention include (meth)acrylic acid or the alkali metal salts and the amine salts thereof; itaconic acid or the alkali metal salts and the amine salts thereof; allylamine or the halogenated hydroacid salts thereof; 3-vinylpropionic acid or the alkali metal salts and the amine salts thereof; vinylsulfonic acid or the alkali metal salts and the amine salts thereof; styrene sulfonic acid or the alkali metal salts and the amine salts thereof; 2-sulfoethylene(meth)acrylate and 3-sulfopropylene (meth)acrylate or the alkali metal salts and the amine salts thereof; 2-acrylamide-2-methylpropane sulfonic acid or the alkali metal salts and the amine salts thereof; acid phosphooxypolyoxyethylene glycol mono(meth)acrylate or the salts thereof; 2-dimethylaminoethyl(meth)acrylate or the halogenated hydroacid thereof; 3-trimethylammoniumpropyl(meth)acrylate; 3-trimethylammoniumpropyl(meth)acrylamide; N,N,N-trimethyl-N-(2-hydroxy-3-methacryloyloxypropyl)ammonium chloride. Also usable as the hydrophilic monomer are 2-hydroxyethyl(meth)acrylate, (meth)acrylamide, N-monometylol(meth)acrylamide, N-dimethylol(meth)acrylamide, N-vinyl pyrrolidone, N-vinyl acetamide and polyoxyethyleneglycol mono(meth)acrylate.

(b) Method for Forming Crosslinked Layer to Which Graft Chains are Introduced

The crosslinked layer to which the graft chains are introduced may be formed by synthesizing a graft polymer using a conventionally known method for producing a graft polymer, followed by cross-linking the resultant graft polymer. Synthesis of a graft polymer is described in “Graft Polymerization and Its Application” written by Humio Ide, edited by Publishing Association of Polymer and published in 1977 by Kyoritsu Publishing Co., and in “New Experimental Polymer Science vol. 2; Synthesis and Reaction of Polymer”, edited by Polymer Science Association, Kyoritsu Publishing Co., 1995.

Basically, synthesis of the graft polymer may be divided into three methods: 1) polymerizing a branch monomer from a backbone polymer; 2) binding a branch polymer to a backbone polymer; and 3) copolymerizing a branch polymer to a backbone polymer (“macromer method”). While any methods described above may be employed for forming the solid support to which the graft polymer is bonded according to the invention, the third method (macromer method) is excellent in view of suitability for production and control of the film structure. Synthesis of the graft polymer using the macromer is described in “New Experimental Polymer Science, vol. 2; Synthesis and Reaction of Polymer”, edited by Polymer Science Association, Kyoritsu Publishing Co., 1995 described above. The method is also described in detail in Isamu Yamashita et al., “Chemistry and Industry of Macromonomer”, I.P.C., 1989.

Specifically, a macromer can be synthesized through a known method using the aforementioned monomers, such as acrylic acid, acrylamide, 2-acrylamide-2-methylpropane sulfonic acid and N-vinyl acetamide, so as to form an organic crosslinked layer.

Examples of the macromer carrying a reactive functional group suitably used in the invention include a macromer derived from a carboxylic group-containing monomer such as acrylic acid and methacrylic acid; a sulfonic acid-type macromer derived from monomers of 2-acrylamide-2-methylpropane sulfonic acid and styrene sulfonic acid, and the salts thereof; an amide-type macromer such as acrylamide and methacrylamide; an amide-type macromer derived from N-vinylcarboxylic acid amide monomers such as N-vinylacetamide, N-vinylfolmamide; a macromer derived from a hydroxyl group-containing monomer such as hydroxyethyl methacrylate, hydroxyethyl acrylate and glycerol monomethacrylate; and a macromer derived from a alkoxy group or ethylene oxide group-containing monomer such as methoxyethyl acrylate, methoxypolyethyleneglycol acrylate and polyethyleneglycol acrylate. Monomers having a polyethyleneglycol chain or a polypropyleneglycol chain may also be used as the macromer for use in the invention.

The macromer usually has a molecular weight of 400 to 100,000, preferably of 1,000 to 50,000, and particularly preferably of 1,500 to 20,000. If the molecular weight is 400 or less, the effect exerted by the macromer cannot be expected. If the molecular weight is larger than 100,000, polymerization with a copolymer constituting the main chain may be impaired.

One method for forming the crosslinked layer to which the graft chains are introduced after synthesis of the macromer comprises the steps of copolymerizing a macromer having a functional group with another monomer having another functional group to synthesize a graft copolymer, and coating on the solid support the resultant graft copolymer together with a crosslinking agent, that is reactive with the functional group in the polymer, to cause a reaction between the synthesized graft copolymer and the reactive functional groups to effect cross-linking by applying heat. Another method comprises the steps of synthesizing a macromer carrying a reactive functional group and a graft polymer having a group capable of photo-crosslinking or a polymerizable group, coating them onto the solid support to effect crosslinking by light irradiation.

As stated above, the surface containing the graft polymer chain can be provided on the substrate. The thickness of the crosslinked layer may be selected depending on the use purposes. Usually, the thickness is preferably 0.001 μm to 10 μm, more preferably 0.01 μm to 5 μm, and most preferably 0.1 μm to 2 μm. When the layer is too thin, scratch resistance may be impaired, while when the layer is too thick, the rate of immobilizing reaction is likely to be lowered.

### EXAMPLES

The present invention will now be explained in more detail by way of the examples below. It should be noted that the invention is not limited to the following examples.

### Example 1

Introduction of a Graft Polymer into a Solid Support

The following photo-polymerizable composition was coated, by means of a rod bar No.17, on a slide glass (slide (A): 25 mm×75 mm) and dried at 80° C. for 2 minutes. The coated layer was pre-cured by irradiation with light for 10 minutes using a 400W high-pressure mercury lamp (UVL-400P manufactured by Rikoh Kagaku Sangyo Co., Ltd.).

Photo-polymerizable composition

| | | |
|---|---|---|
| Allyl methacrylate/methacrylic acid copolymer | 4 | g |
| (molar ratio:80/20, molecular weight: 100,000) | | |
| Ethylene oxide-modified bisphenol A diacrylate | 4 | g |
| (M210 manufactured by Toa Gosei Chemical Industry Co.) | | |
| 1-Hydroxycyclohexyl phenyl ketone | 1.6 | g |
| 1-Methoxy-2-propanol | 6 | g |

Then, the resultant slide glass (A) was immersed in an aqueous solution containing 10% by mass of acrylic acid and 0.01% by mass of sodium hypochlorite and irradiated with light for 30 minutes using a 400W high-pressure mercury lamp in an argon atmosphere.

After irradiation, the slide glass was thoroughly washed with ion-exchanged water to prepare a solid support (slide (B)) to which acrylic acid was grafted on the surface thereof.

Conversion of Acrylic Acid into an Active Ester

In order to convert the carboxyl group at a terminal end of acrylic acid into an active ester, the slide (B) in which acrylic acid was introduced into the graft chain was immersed in an acetonitrile (50 mL) solution containing 4% by mass of 1-ethyl-3-(3-dimethylaminopropyl)carbodiimide hydrochloride (manufactured by Tokyo Kasei Kogyo Co., Ltd.) and 10% by mass of N-hydroxysuccinimide for 2 hours, washed with acetonitrile and then dried under reduced pressure for one hour, to thereby prepare a slide (C) into which a succinimide group was introduced.

Spot-Wise Application of DNA Fragments and Measurement of Fluorescence Intensity

An aqueous solution (1×10⁻⁶M, 1 μL) in which DNA fragments carrying amino groups at both 5′ terminal and 3′ terminal ((5′ terminal→3′ terminal): CAGGCATACACTGAA GTGAAAACTG) and labeled with a fluorescent tag using a labeling reagent (FluoroLink Cy5dCTP, Amerciam Pharmacia Biotech Corporation) were dispersed in a 0.1M carbonate buffer solution (pH:9.8) was applied spot-wise to the above obtained slide (C). After the spot-wise application, the resultant slide was allowed to stand at 40° C. and 90% humidity for one hour. Then the slide was successively washed twice with a mixture of a 0.1% by mass of SDS (sodium dodecyl sulfate) and 2×SSC (a 10-fold dilution of a standard saline citrate buffer solution (20×SSC: containing 3.0M NaCl and 0.3M sodium citrate)) and once with a 0.2×SSC aqueous solution (a 100-fold dilution of a standard saline citrate buffer solution (20×SSC)). Then, the resultant slide was dried at room temperature to thus produce a slide (D1) having DNA fragments immobilized on the surface thereof, i.e., a DNA chip according to the invention.

The surface of the thus produced slide (D1) was measured for fluorescence intensity using a fluorescence scanning apparatus. A value of 1500 was obtained, confirming that the DNA fragments were efficiently immobilized on the slide glass by the immobilizing method of the invention.

### Example 2

Assessment of Hybridization

A slide (D′1) (DNA chip) on which DNA fragments were immobilized was produced in the same manner as in Example 1, except that applied DNA fragments were not tagged with the labeling reagent (FluoroLink Cy5dCTP, Amerciam Pharmacia Biotech Corporation).

A solution was prepared by dispersing a 22mer target oligonucleotide (CTAGTCTGTGAAGTTCCAGATC-5′) having Cy5 linked to 5′ terminal in a hybridization liquid (a mixture of 4×SSC and 10% by mass of SDS)(20 μL) and then applied spot-wise to the slide (D′1) produced in Example 1. After the surface was covered with a cover glass used for a microscope, the slide (D′1) was incubated in a moisture chamber at 60° C. for 20 hours. Then, the resultant slide (D′1) was successively washed with a mixture of a 0.1% by mass of SDS and 2×SSC, a mixture of a 0.1% by mass of SDS and 0.2×SSC, and a 0.2×SSC aqueous solution. After the washing, the slide was centrifuged at 600 rpm for 20 second and dried at room temperature. The surface of the slide glass was measured for fluorescence intensity using a fluorescence scanning apparatus. A value of 1200 was obtained, to indicate that fluorescence intensity was considerably increased as compared with the background. The results reveal that the target DNA fragment, which was contained in a sample and complementary to the DNA fragment immobilized on the DNA chip, can be effectively detected using the DNA chip of the invention.

### Example 3

Preparation of a Solid Support into Which a Graft Polymer is Introduced

A biaxially oriented polyethylene terephthalate film (A4100, manufactured by Toyobo Co., Ltd.), having a thickness of 188 μm, was subjected to an oxygen glow treatment using a planographic magnetron sputtering apparatus (CFS-10-EP70, manufactured by Shibaura Eletec Corporation) under the following conditions.

<Oxygen Glow Treatment Conditions>

Initial vacuum: 1.2×10⁻³ Pa

Oxygen pressure: 0.9 Pa

RF glow: 1.5 KW

Treating time: 60 sec

The film, which had been subjected to the glow treatment, was immersed in a nitrogen bubbled solution (10 wt %) of acrylic acid at 70° C. for 7 hours. After the immersion, the film was washed with water for 8 hours to thereby prepare a solid support (solid support 1) in which acrylic acid was graft-polymerized on the surface thereof.

Conversion of Acrylic Acid into an Active Ester

In order to convert the carboxyl group at a terminal end of acrylic acid into an active ester, solid support 1 to which acrylic acid was introduced into the graft chain was immersed in an acetonitrile (50 mL) solution containing 4% by mass of 1-ethyl-3-(3-dimethylaminopropyl)carbodiimide hydrochloride (manufactured by Tokyo Kasei Kogyo Co., Ltd.) and 10% by mass of N-hydroxysuccinimide for 2 hours, washed with acetonitrile and then dried under reduced pressure for one hour, to thereby prepare a solid support into which a succinimide group was introduced as the reactive functional group.

Spot-Wise Application of DNA Fragments and Measurement of Fluorescence Intensity

An aqueous solution (1×10⁻⁶M, 1 μL) in which DNA fragments carrying amino groups at both 3′ terminal and 5′ terminal ((5′ terminal→3′ terminal):CAGGCATACACTGAA GTGAAAACTG) and labeled with a fluorescent tag using a labeling reagent (FluoroLink Cy5dCTP, Amerciam Pharmacia Biotech Corporation) were dispersed in a 0.1M carbonate buffer solution (pH:9.8) was applied spot-wise to solid support 1 obtained above. After the spot-wise application, the resultant solid support was allowed to stand at 40° C. and 90% humidity for one hour. Then solid support 1 was successively washed twice with a mixture of a 0.1% by mass of SDS (sodium dodecyl sulfate) and 2×SSC (a 2-fold dilution of a standard saline citrate buffer solution (SSC)) and once with a 0.2×SSC aqueous solution. Then, the resultant support was dried at room temperature to thus produce support 1 having DNA fragments immobilized on the surface thereof, i.e., a DNA chip of Example 3.

This DNA chip was evaluated for fluorescence intensity using a fluorescence scanning apparatus at the surface thereof. A value of 1500 was obtained, confirming that the DNA fragments were efficiently immobilized on a PET film, serving as the solid support, by the immobilizing method of the invention.

### Example 4

A DNA chip of Example 4 was produced in the same manner as in Example 3, except that a cellulose acetate butyrate film (having a thickness of 200 μm) was used in place of the biaxially oriented polyethylene terephthalate film.

As in Example 3, DNA fragments were immobilized on the support and the produced DNA chip was measured for fluorescence intensity using a fluorescence scanning apparatus at the surface thereof. The obtained value was 1600, confirming that the DNA fragments were efficiently immobilized on the solid support even if the resinous material for forming the solid support was changed to another resinous material in the DNA chip of the invention.

As stated above, the DNA chip of the invention has high DNA-adsorbing efficiency enabling immobilization of a large number of DNA fragments per unit area of the chip to be useful enough in analyzing the function of genes in an organism. This DNA chip provides advantages that hybridization occurs effectively thereon and hence a target DNA fragment, contained in a sample and complementary to a probe DNA fragment immobilized on the DNA chip, is efficiently detected.

Further, the DNA chip of the invention comprising the solid support made of a resinous material has dense active sites to immobilize a large number of DNA fragments per unit area and thereby achieve good DNA bonding.

What is claimed is:

1. A DNA chip comprising:
a solid support having a surface;
a DNA fragment immobilized on said surface; and
a graft polymer bonded to said surface, wherein the DNA fragment is immobilized on said surface via said graft polymer.

2. The DNA chip according to claim 1, wherein the solid support is made of an inorganic material selected from the group consisting of glass, cement, ceramics, new ceramics, silicon, activated carbon and gold.

3. The DNA chip according to claim 1, wherein the solid support has a shape selected from the group consisting of a plate, a porous substance, a fabric, a knit and a non-woven fabric.

4. The DNA chip according to claim 3, wherein the porous substance is selected from the group consisting of porous glass, porous ceramics, porous silicon, porous activated carbon and a membrane filter.

5. The DNA chip according to claim 1, wherein the solid support has a thickness of 100 μm to 2,000 μm.

6. The DNA chip according to claim 1, wherein the graft polymer has, in a side chain thereof, a reactive functional group capable of bonding with the DNA fragment.

7. The DNA chip according to claim 6, wherein the reactive functional group is selected from the group consisting of a carboxyl group, an amino group, a hydroxyl group, an epoxy group, an acid anhydride group, a sulfonic acid group, an isocyanate group, a trialkoxysilyl group, a thioisocyanate group, a phosphoric acid group and a phosphonic acid group.

8. The DNA chip according to claim 1, wherein the graft polymer comprises a chain having a molecular weight of 500 to 5,000,000.

9. The DNA chip according to claim 1, wherein the graft polymer is formed by surface graft polymerization.

10. The DNA chip according to claim 1, wherein the graft polymer has a construction in which a graft portion, that has the reactive functional group for interacting with the DNA fragment, is substantially un-crosslinked.

11. A DNA chip comprising:
a solid support made of a resinous material and having a surface;
a DNA fragment immobilized on said surface; and
a graft polymer bonded to said surface, wherein the DNA fragment is immobilized on said surface via said graft polymer.

12. The DNA chip according to claim 11, wherein the resinous material is a plastic film.

13. The DNA chip according to claim 12, wherein the plastic film is made of a resin selected from the group consisting of cellulose acetate butyrate, cellulose triacetate, cellulose tributyrate, polyacetal, polyamide, polyamideimide, polyarylate, polyimide, polyetherimide (PEI), polyetheretherketone (PEEK), polyethersulfone (PES), polyethylene terephthalate (PET), polyethylene naphthalate (PEN), polycarbonate, polysulfone (PSF), polystyrene, polycellulose triacetate, polyphenylene sulfide (PPS), polyphenylene oxide (PPO), polybenzoxazole and amorphous polyacrylate (PAR).

14. The DNA chip according to claim 11, wherein the resinous material is a non-fluorescent plastic film selected from the group consisting of polymethyl methacrylate, cellulose acetate butyrate, cellulose triacetate, cellulose tributyrate, polyacetal and polyamide.

15. The DNA chip according to claim 11, wherein the resinous material is selected from the group consisting of an epoxy resin, an acrylic resin, an urethane resin, a phenolic resin, a styrene-type resin, a vinyl-type resin, a polyester resin, a polyamide-type resin, a melamine-type resin and a formalin resin.

16. The DNA chip according to claim 11, wherein the graft polymer has, in a side chain thereof, a reactive functional group capable of bonding with the DNA fragment.

17. The DNA chip according to claim 16, wherein the reactive functional group is selected from the group consisting of a carboxyl group, an amino group, a hydroxyl group, an epoxy group, an acid anhydride group, a sulfonic acid group, an isocyanate group, a trialkoxysilyl group, a thioisocyanate group, a phosphoric acid group and a phosphonic acid group.

18. The DNA chip according to claim 11, wherein the graft polymer comprises a chain having a molecular weight of 500 to 5,000,000.

19. The DNA chip according to claim 11, wherein the graft polymer is formed by surface graft polymerization.

20. The DNA chip according to claim 11, wherein the graft polymer has a construction in which a graft portion, that has the reactive functional group for interacting with the DNA fragment, is substantially un-crosslinked.


SEQUENCE LISTING (2 sequences)

SEQ ID NO: 1 - DNA, 25 bp, Artificial Sequence
  test sequence
caggcatacactgaagtgaaaactg

SEQ ID NO: 2 - DNA, 22 bp, Artificial Sequence
  target oligonucleotide
ctagaccttgaagtgtctgatc